(19) United States
(12) Patent Application Publication
(10) Pub. No.: US 2021/0052494 A1
(43) Pub. Date: Feb. 25, 2021

(54) BIO-REMEDIAL NON-IMMUNOGENIC CANNABINOID DELIVERY

(71) Applicant: Sorse Technology Corporation, Seattle, WA (US)

(72) Inventor: Michael Flemmens, Seattle, WA (US)

(21) Appl. No.: 17/001,250

(22) Filed: Aug. 24, 2020

Related U.S. Application Data
(60) Provisional application No. 62/891,179, filed on Aug. 23, 2019.

(51) Int. Cl. A61K 9/107; A61K 9/00; A61K 31/05; A61K 47/34; A61K 31/352

(52) U.S. Cl. CPC A61K 9/107; A61K 9/0014; A61K 31/352; A61K 9/0019; A61K 47/34; A61K 31/05

(57) ABSTRACT

A method for manufacturing a bio-remedial non-immunogenic cannabinoid delivery mechanism is provided. An alcohol monomer and an acid monomer are combined. One or more cannabinoids are added to the combination of alcohol monomer and acid monomer. A polymeric material of the cannabinoids, alcohol monomer, and acid monomer is formed and processed into a product for insertion, injection, or topical application by a user.

### CROSS-REFERENCE TO RELATED APPLICATION

This non-provisional patent application claims priority under 35 U.S.C. § 119(e) to U.S. Provisional patent application, Ser. No. 62/891,179, filed Aug. 23, 2019, the disclosure of which is incorporated by reference.

### FIELD

This application relates in general to drug delivery and, in particular, to bio-remedial non-immunogenic cannabinoid delivery.

### BACKGROUND

As states and countries begin to legalize cannabis, much research has been conducted with respect to identifying uses and benefits of cannabis. Particularly, health and medical benefits have been identified, which result from cannabis use. Some health benefits identified include relief of chronic pain, treatment of depression, regulation of seizures, quicker healing time for broken bones, and use as an anti-inflammatory, including for slowing the development of Alzheimers and treatment of inflammatory bowel disease, among other health benefits.

Common methods for cannabis use by users seeking the medical benefits include smoking, vaping, and ingesting food, beverage, or capsulated products that include cannabis, as well as applying topical forms of cannabis, such as lotions, salves, oils, or sprays. However, each current means for ingesting or applying cannabis is a single dose and has a limited time of effectiveness before another dose is needed. Each dose contains a specified amount of cannabis and when additional relief is required, another dose must be taken.

Methods for continuous release of the cannabis would allow the cannabinoids to be effective over a longer period of time than the single doses so that the user doesn't need to keep taking additional doses. However, controlling release of the cannabinoids over an extended period of time requires an appropriate delivery mechanism. Ideally, the delivery mechanism would allow for internal and external release of the cannabinoids with respect to the user's body. For example, a patch would allow for topical application of the cannabinoids, while an implant or capsule would allow for release of the cannabinoids within a user's body.

Poly(glycerol sebacate) (PGS) is a bioabsorbable polymeric material that includes a filament, fiber, yarn, knit material, mesh, a tube or a coating. U.S. Pat. No. 9,359,472 to Nicholson discloses a process for preparing the polymeric material with antimicrobial properties upon degradation of the material using a water mediated process. Specifically, water, glycerol, and sebacic acid are combined, the sebacic acid is melted, the water is distilled, and a reaction of the remaining water, glycerol and sebacic acid react. The resulting product is a resin that is cured into different sizes and shapes. However, such method fails to consider incorporating cannabinoids, which are hydrophobic and thus, water insoluble. Further, generating immediate, extended, and blended release of the cannabinoids is not provided by the current material.

Therefore, a need for a delivery mechanism for controlled release of cannabinoids is needed. Preferably, a reaction process allows the cannabinoids to be incorporated in the material of the delivery mechanism and can be released over time.

### SUMMARY

Preparing a delivery mechanism, such as a polymeric material in which cannabis is incorporated for controlled release, includes mixing cannabinoids with an alcohol monomer and an acid monomer. The cannabinoids can be in the form of an isolate, full spectrum, powder, or liquid, such as an emulsion. The step at which the cannabinoids are added during the processing determines how soon the cannabinoids will be released from the polymeric material upon administration to a wearer. An alcohol monomer and acid monomer are combined with one or more cannabinoids and the molecules react to form the polymeric material via a condensation reaction. When the cannabinoids are added at the beginning of the process, such as with the alcohol and acid monomers, the cannabinoids can be added into a backbone of the structure for the material for an extended release time. However, when the cannabinoids are later added, less cannabinoids are bound to the final product and many of the cannabinoids remain free or bound via weaker bonds so that the crosslink density of the material is low and the cannabinoids can be immediately or more quickly released.

An embodiment provides a method for manufacturing a bio-remedial non-immunogenic cannabinoid delivery mechanism. An alcohol monomer and an acid monomer are combined. One or more cannabinoids are added to the combination of alcohol monomer and acid monomer. A polymeric material of the cannabinoids, alcohol monomer, and acid monomer is formed and processed into a product for insertion, injection, or topical application by a user.

A further embodiment provides a method for preparing a polymeric material. An alcohol monomer and an acid monomer are combined. An emulsion comprising one or more cannabinoids is to the combination of alcohol monomer and acid monomer. A polymeric material of the emulsified cannabinoids, alcohol monomer, and acid monomer is formed and processed into a product for insertion, injection, or topical application by a user.

A still further embodiment provides a polymeric material, including a plurality of alcohol monomer molecules and a plurality of acid monomer molecules. One or more cannabinoids in a form of an emulsion are added to the alcohol monomer molecules and acid monomer molecules. The alcohol monomer molecules, acid monomer molecules, and cannabinoids are combined in a molar ratio of 0.5 to 5 mol alcohol monomer molecules, 0.5 to 5 mol acid monomer molecules, and 0.1% to 100% of 0.5 to 5 mol emulsion. The alcohol monomer molecules, acid monomer molecules, and cannabinoids are crosslinked via condensation reactions to form a polymeric material

Still other embodiments will become readily apparent to those skilled in the art from the following detailed description, wherein are described embodiments by way of illustrating the best mode contemplated. As will be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

### BRIEF DESCRIPTION OF THE DRAWINGS

FIG. 1 is a flow diagram showing a method for processing a bio-remedial non-immunogenic cannabinoid delivery mechanism.

FIG. 2 is a block diagram showing, by way of example, a density of crosslinking between the cannabinoids and the glycerol and sebacic acid.

### DETAILED DESCRIPTION

Poly(glycerol sebacate) (“PGS”) is a polymeric material that is used in medical applications and is manufactured with two monomers that are metabolites. See, Y Wang, S. Lu, P. Gabriel, J. Harris, “Poly (Glycerol Sebacate) in Tissue Engineering and Regenerative Medicine,” Material Matters, 2016, 11.3 and U.S. Pat. No. 9,359,472 to Nicholson, which is hereby incorporated by reference in its entirety. The material is manufactured through a condensation reaction where a hydroxyl group (OH) and hydrogen (H) side chains react and kick off water. PGS is non-immunogenic as the material and its constituent building blocks are common metabolites available in the metabolic pathways. PGC also has antimicrobial properties to ensure safety when medically applied.

Cannabis, which has been shown to have multiple medical benefits, can be incorporated into the PGS material for controlled release, which occurs when the PGS material degrades, such as upon the application of water or other liquid. Specifically, most if not all, cannabinoids contain OH functionality and can be crossed linked into the PGS material formed from glycerol and sebacic acid. The stage of processing during which cannabinoids are added controls the time of release, and when added at multiple stages, the cannabinoids can include both immediate and extended release, which can offer a wearer longer relief from pain, longer periods of seizure regulation, and a reduction in inflamed body parts. For example, the PGS material is reversibly degraded via hydrolysis, eventually releasing any bound cannabinoids and the cannabinoids first introduced to the process will be released later than cannabinoids later introduced during processing.

The concept is to create immediate, extended, and blended release products by combining cannabinoids with glycerol and sebacic acid, generally used to form PGS. For example, some native CBD can be incorporated into the backbone of the material during synthesis. This builds an extended release material. Protected (emulsified liquid, or encapsulated powder) incorporation into a finished PGS matrix would provide more of an immediate release. Blending the two results in a sustained release and an initial loading dose to get the patient into a therapeutic window.

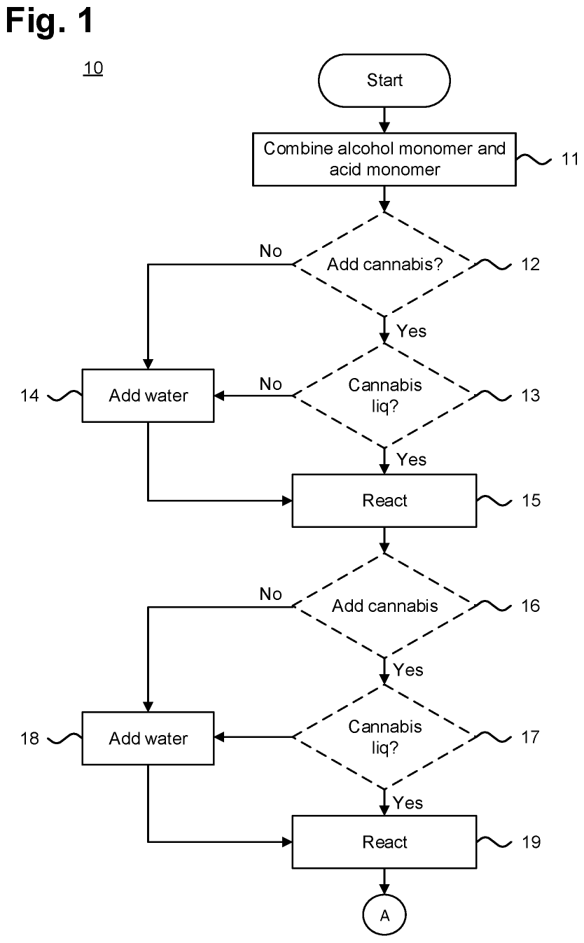

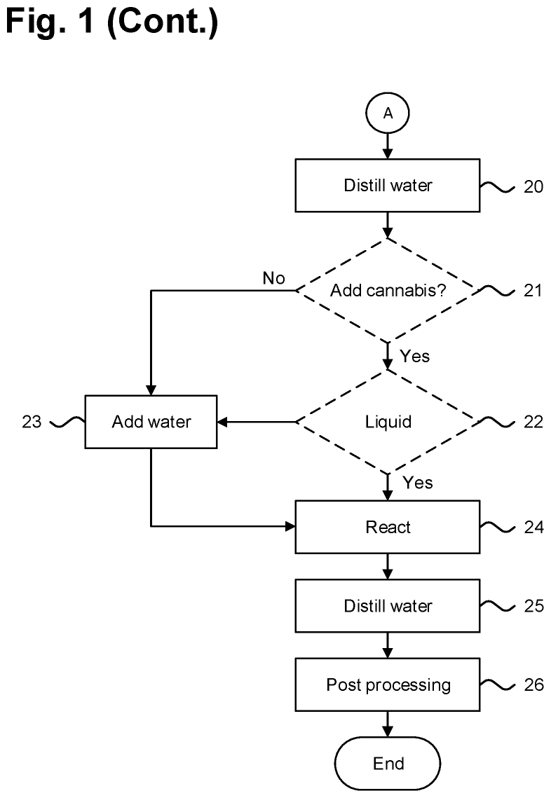

The process for forming a polymeric material for controlled cannabis release can include the addition of the cannabinoids in different forms, including emulsion, powder, isolate or full spectrum forms. FIG. 1 is a flow diagram showing a method for processing a bio-remedial non-immunogenic cannabinoid delivery mechanism. An alcohol monomer and acid monomer are combined (step 11). Specifically, a plurality of alcohol monomer molecules are combined with a plurality of acid monomer molecules to perform crosslinking, as described below. The alcohol monomer can be glycerol and the acid monomer can be sebacic acid. However, other alcohol and acid monomers are possible, such as those described in U.S. Pat. No. 9,359,472 to Nicholson, which is hereby incorporated by reference.

One or more cannabinoids extracted from the cannabis plant can also be optionally added (step 12) to the glycerol and sebacic acid. The cannabinoids can be in different forms and can include Cannabigerolic acid, Δ9-tetrahydrocannabinolic acid (THCA), Cannabidiolic acid, Cannabichromenenic acid, Cannabigerovarinic acid, Tetrahydrocanabivarinic acid, Cannabidivarinic acid, Cannabichromevarinic acid, Cannabigerol, all forms of Tetrahydrocannabinol (THC), including Δ9-tetrahydrocannabinol and Δ8-tetrahydrocannabinol, Cannabidiol, Cannabichromene, Cannabigerivarin, Tetrahydrocannabivarin, Cannabidivarin, Cannabichromevarin, Cannabinol, and other cannabinoids. At a minimum, the cannabinoid should have an available hydroxyl group for binding.

If the cannabinoids are in the form of a liquid (step 13), such as an emulsion, the liquid can be added directly to the glycerol and sebacic acid without any water or other component, and heated to allow cross linking of the glycerol, sebacic acid and cannabinoid molecules. The combination can be heated to a temperature between 20° C.-200° C. for a time period of around 1 to 336 hours.

The liquid can be an emulsification of the cannabinoids, which is prepared according to the process described in detail in commonly-owned U.S. patent application Ser. No. 16/010,082, to Riefler, which is hereby incorporated by reference. Since cannabinoids are often extracted in oil form and are hydrophobic, emulsifying the cannabinoids evenly distributes the oil droplets throughout water for use in products, such as ingestible and topical products. When the cannabinoids in emulsion form are combined with glycerol and sebacic acid, no other components or molecules are necessary. The liquid emulsion can be used in lieu of water to melt the sebacic acid when in solid form. Subsequently, any remaining water can be distilled.

However, if the cannabinoids are in a powder form, water can be added to dissolve the powder. The powder can be a dried form of the emulsified cannabinoids processed according to the method also described in U.S. patent application Ser. No. 16/010,082, to Riefler. The cannabinoids can be in full spectrum or isolate form. Upon liquification of the sebacic acid, the water can be distilled.

The glycerol, sebacic acid, and cannabinoids react (step 15) and polymerize to form a polymeric material with cannabinoids bound into the backbone of the material. Specifically, the cannabinoids can bind with the hydroxyl groups on the glycerol and sebacic acid via a condensation reaction, which occurs at a range of temperatures. For example, the glycerol, sebacic acid, and cannabinoids are heated to a temperature within a range of about 50° C. to 200° C. In one example, the temperature range can be 80° C. to 150° C. However, other temperatures and time periods are possible. The heating can also occur under inert gas. During the reaction, kinetic crosslinking occurs and the glycerol, sebacic acid, and cannabinoids randomly bind over time. Structures of the molecules are shown below:

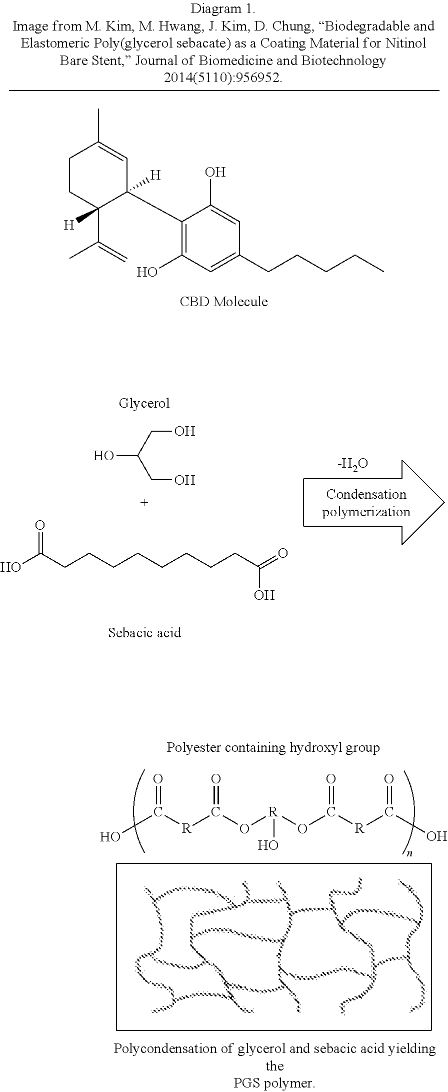

The longer the reaction is allowed to occur, the higher percentage of crosslinking of the glycerol, sebacic acid, and cannabinoids occurs. For example, the polymerization can continue for a range of about 1 hour to 336 hours. In one embodiment, the time range is around 12 hours to 168 hours.

When the cannabinoids are in a more viscous form than liquid (step 13), such as a powder, water is added (step 14) to reduce the viscosity of the cannabinoids. The amount of water can vary based on the viscosity with more water required for more viscous forms. If water is added, any remaining water can be distilled. Subsequently, the crosslinking occurs (step 15) to form the polymeric material.

However, if no cannabinoids are added to the glycerol and sebacic acid at the initial stage, water is added in lieu to melt the sebacic acid. After melting of the sebacic acid, the water is distilled and the glycerol and sebacic acid can react and begin to polymerize to form PGS at temperatures within a particular range, as described above. The glycerol, sebacic acid, and water can be combined in a molar ratio of about 0.5 to 5 mol glycerol, 0.5 to 5 mol sebacic acid, and 0.5 to 5 mol water, as described in detail in U.S. Pat. No. 9,359,472, which is hereby incorporated by reference. In one embodiment, the ranges can be 1-2 mol glycerol, 1-2 mol sebacic acid, and 2-5 mol water in molar ratio.

However, when cannabinoids in emulsion form are added in lieu of water, as described above, the amount of the emulsion can equal 0.1% to 100% of the amount of water used in lieu of cannabis, as described above. In a further embodiment, the emulsion can be mass balanced to equal the amount of water. For instance, the water ratio in the emulsion can be 70%. An emulsion weighing 100 g can include an additional 30 g of water to make up the difference for the 70% water in the emulsion, as compared to when water is used alone. Further, additional emulsion can be added to make up the difference, which in this example, would be around 42.9 g of cannabinoid emulsion. Also, when less emulsion is used, water or another aqueous liquid can be used to equal 0.5 to 5 mol ratio with respect to 0.5-5 mol glycerol and 0.5-5 mol sebacic acid.

Further, the amount of cannabinoids in the emulsion can be dependent on the emulsifying agent. For example, Gum Arabic, Gum Acacia, and modified food starch, can be used at a ratio of 1 part cannabinoid oil mixture to 0.1 to 4.0 parts emulsifying agent. Meanwhile, mono and/or diglycerides, Tween 20 or 80, and Q-Natural could be used at lower levels ranging from 1 part oil mixture to 0.1 to 1.0 emulsifying agent. The type of emulsifying agent also has an effect on how the cannabinoids bind to the glycerol or sebacic acid. For example, Gum Arabic enrobes the cannabinoid and fails to leave the hydroxyl group available for bonding with the glycerol or sebacic acid. Accordingly, the Gum Arabic itself binds to the glycerol or sebacic acid and holds the cannabinoid in place. In contrast, other emulsifying agents enrobe the cannabinoid, but leave the hydroxyl group available for binding.

In a further embodiment, the emulsion can include glycerol as a diluent, cannabinoids, water, and an emulsifying agent. Subsequently, the emulsion can be mixed only with the sebacic acid, not the glycerol or water, to form the polymeric material. Performing the emulsification with glycerol as a diluent is described in detail in U.S. patent application Ser. No. 16/010,082, to Riefler, which is hereby incorporated by reference.

Regardless of whether cannabinoids are initially added with the glycerol and sebacic acid, cannabinoids can also be added (step 16) during the polymerization of glycerol and sebacic acid, but to completion of the polymerization. Different forms of the cannabinoids can be added using the method as described above. If the cannabinoids are a liquid (step 17), only the emulsified cannabinoids are added to the glycerol and sebacic acid, without any addition of water. However, if the cannabinoids are in a powder form, water can be added (step 18) before reaction of the cannabinoids, and glycerol and sebacic occurs (step 19).

When the cannabinoids are introduced after the glycerol and sebacic have had some opportunities to cross link, the cannabinoids still bind to the hydroxyl groups of the glycerol or sebacic acid, but are generally bound on ends of the molecule chains, rather than integrated into the background of the polymeric material, such as when the cannabinoids are introduced with the glycerol and sebacic acid. Further, since the time for reaction is generally less, due to being added during an intermediate stage, some cannabinoids remain free or form weaker bonds with the glycerol and sebacic acid. Below is an example of the crosslinking between glycerol and sebacic acid, which occurs prior to introduction of the cannabinoids. Once introduced, the cannabinoids bind to the available hydroxyl groups, such as on the glycerol.

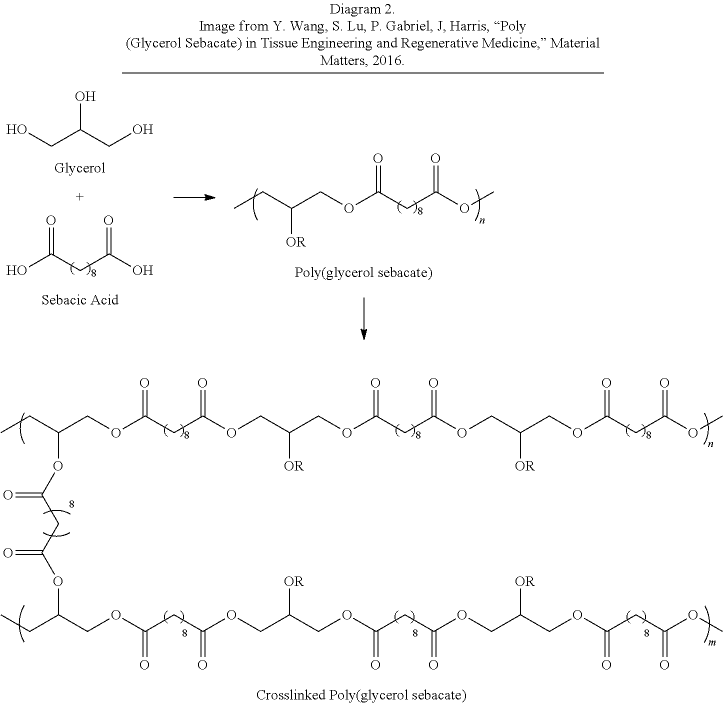

After crosslinking has occurred, the water generated via the condensation reactions between the glycerol, sebacic acid and cannabinoids is distilled (step 20).

Further, cannabinoids can also be added (step 21) after the glycerol and sebacic acid have fully crosslinked. If the cannabinoids are a liquid (step 22), the cannabinoids bind to the open hydroxyl groups on the crosslinked glycerol and sebacic acid. If the cannabinoids are in a more viscous form, water is added (step 23), the cannabinoids bind to the crosslinked glycerol and sebacic acid molecules (step 24), and remaining water is distilled (step 25). The amount of cannabinoids that bind to the glycerol and sebacic acid depend on the time allowed for the reaction to occur. The shorter the time, the less cannabinoids that bind. Accordingly, when the cannabinoids are added after crosslinking of the glycerol and sebacic acid, many cannabinoids remain free or are weakly bound to the glycerol and sebacic acid. After, post processing of the polymeric material can occur, as described below. If no cannabinoids are added (step 21), the polymeric material is completed and can be post processed (step 26) to form particular products.

Post processing can include forming implantable devices, patches, and wound care devices, as well as other types of products. For example, an implantable tube can be formed from the polymeric material by curing the material in a forming template. The tube can be inserted in a user and once implanted, the tube is in contact with bodily fluids and heat that help drive hydrolysis to degrade the tube to release the cannabinoids. Other types of implantable devices can include adhesion barriers, drug delivery rods, and mesh coatings—leveraging any cannabinoid therapeutic properties including but not limited to anti-inflammatory.

Another example includes forming the polymeric material into a patch for application on the skin of a wearer. The viscous polymeric material is poured into a form and cured. The time cured determines how hard the material becomes with longer cure times increasing the hardness of the material. Subsequently, the wearer places the patch on his skin and the moisture from the wearer's skin and body heat drives hydrolysis of the patch, which degrades over time to release the cannabinoids. The patch can also be used in the wearer's mouth and the wearer's saliva can degrade the patch over time, thereby releasing the cannabinoids. In yet further examples, the viscosity of the polymeric material can be increased or thinned to generate a spray, lotion, or other topical product for application. Topicals can be targeted to immune system driven skin conditions, such as psoriasis.

Additionally, the polymeric material can be formed into capsules or other products for ingestion by the user, as well as a powder. For a powder, the polymeric material can be fully crosslinked and subsequently, cryo-ground to manufacture the powder. Any molecular weight or viscosity of the polymeric material can be manufactured to create different types of products, some of which are discussed above.

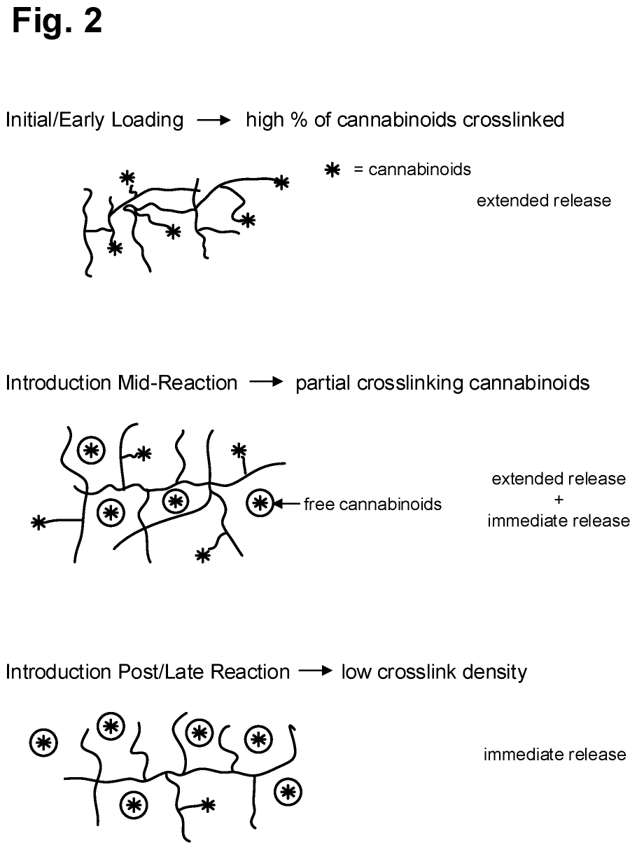

The rate of release of the cannabinoids depends on the particular stage of the polymerization process that the cannabinoids are added. FIG. 2 is a block diagram showing, by way of example, a density of the crosslinking between the cannabinoids and the glycerol and sebacic acid. When the cannabinoids are introduced initially with the glycerol and sebacic acid, there is a high percentage of the cannabinoids that crosslink with the open hydroxyl groups of the glycerol and sebacic acid via a condensation reaction, which creates an extended period time for release of the cannabinoids upon degradation of the polymeric material. In one example, the cannabinoids can be released gradually over a period of around 6 to 12 months. However, other time periods are possible.

When the cannabinoids are introduced mid-reaction, there can be partial crosslinking of the cannabinoids with the glycerol and sebacic acid to allow some cannabinoids to bind via hydrolysis and others to remain free or connected via weaker bonds. When the polymeric material degrades, the cannabinoids that remain free or have weaker bonds are released more immediately than those cannabinoids that are bound to the glycerol and sebacic acid via condensation reactions or that form part of the backbone of the polymeric material with the glycerol and sebacic acid. The more immediate release of the cannabinoids can occur within hours. Finally, when the cannabinoids are introduced after PGS is formed via crosslinking of the glycerol and sebacic acid, there is a low percentage of crosslinking between the introduced cannabinoids and the glycerol and sebacic acid. Further, cannabinoids can be introduced during two or more of the processing stages to generate a polymeric material with continuous release of the cannabinoids.

Some cannabinoids provide an anti-inflammatory response, while PGS itself also helps to reduce inflammation. When combined, the polymeric material can provide an increased anti-inflammatory response, among other health benefits as a result of cannabinoid use.

The cannabinoids, glycerol, and sebacic acid can be combined in a vessel in which the reaction occurs in dose batches. Alternatively, an extruder can be used for continuous production of the polymeric material, rather than batch processing as in the vessel. For example, the sebacic acid can be poured into an extruder and continuously dosed into a hopper. The cannabinoids and glycerol can then be added. Further, the cannabinoids can be added at different stages of processing and the combination can be moved to different sections to allow for combining, heating, and reactions so that each holding section is at a different processing stage.

While the invention has been particularly shown and described as referenced to the embodiments thereof, those skilled in the art will understand that the foregoing and other changes in form and detail may be made therein without departing from the spirit and scope of the invention.

What is claimed is:

1. A method for manufacturing a bio-remedial non-immunogenic cannabinoid delivery mechanism, comprising:
combining an alcohol monomer and an acid monomer;
adding one or more cannabinoids to the combination of alcohol monomer and acid monomer; and
forming a polymeric material of the cannabinoids, alcohol monomer, and acid monomer into a product for insertion, injection, or topical application by a user.

2. A method according to claim 1, wherein the cannabinoids are one of emulsified and in powder form.

3. A method according to claim 1, wherein the cannabinoids comprise one or more of Cannabigerolic acid, Δ9-tetrahydrocannabinolic acid (THCA), Cannabidiolic acid, Cannabichromenenic acid, Cannabigerovarinic acid, Tetrahydrocanabivarinic acid, Cannabidivarinic acid, Cannabichromevarinic acid, Cannabigerol, all forms of Tetrahydrocannabinol (THC), including Δ9-tetrahydrocannabinol and Δ8-tetrahydrocannabinol, Cannabidiol, Cannabichromene, Cannabigerivarin, Tetrahydrocannabivarin, Cannabidivarin, Cannabichromevarin, and Cannabinol.

4. A method according to claim 1, wherein the reaction comprises condensation reactions.

5. A method according to claim 4, wherein water produced as a by-product of the condensation reactions is removed via distillation.

6. A method according to claim 1, wherein the alcohol monomer comprises glycerol.

7. A method according to claim 1, wherein the acid monomer comprises sebacic acid.

8. A method according to claim 1, wherein the cannabinoids are added with the alcohol monomer and acid monomer before crosslinking of the alcohol monomer and acid monomer has occurred, after crosslinking has started, and after crosslinking is complete.

9. A polymeric material prepared according to the method of claim 1.

10. A method for preparing a polymeric material, comprising:
combining an alcohol monomer and an acid monomer;
adding an emulsion comprising one or more cannabinoids to the combination of alcohol monomer and acid monomer at an initial time or a subsequent time after reaction of the glycerol and sebacic acid; and
forming a polymeric material of the cannabinoids, alcohol monomer, and acid monomer into a product for insertion, injection, or topical application by a user.

11. A method according to claim 10, further comprising:
heating the combination of alcohol monomer, acid monomer, and emulsion.

12. A method according to claim 10, wherein the cannabinoids comprise one or more of Cannabigerolic acid, Δ9-tetrahydrocannabinolic acid (THCA), Cannabidiolic acid, Cannabichromenenic acid, Cannabigerovarinic acid, Tetrahydrocanabivarinic acid, Cannabidivarinic acid, Cannabichromevarinic acid, Cannabigerol, all forms of Tetrahydrocannabinol (THC), including Δ9-tetrahydrocannabinol and Δ8-tetrahydrocannabinol, Cannabidiol, Cannabichromene, Cannabigerivarin, Tetrahydrocannabivarin, Cannabidivarin, Cannabichromevarin, and Cannabinol.

13. A method according to claim 10, wherein the reaction comprises condensation reactions.

14. A method according to claim 13, wherein water produced as a by-product of the condensation reactions is removed via distillation.

15. A method according to claim 10, wherein the alcohol monomer comprises glycerol.

16. A method according to claim 10, wherein the acid monomer comprises sebacic acid.

17. A method according to claim 10, wherein the cannabinoids are combined with the alcohol monomer and acid monomer before crosslinking of the alcohol monomer and acid monomer has occurred, after crosslinking has started, and after crosslinking is complete.

18. The polymeric material prepared according to the method of claim 10.

19. A polymeric material, comprising:
a plurality of alcohol monomer molecules;
a plurality of acid monomer molecules; and
one or more cannabinoids in a form of an emulsion,
wherein the alcohol monomer molecules, acid monomer molecules, and cannabinoids are combined in a molar ratio of 0.5 to 5 mol alcohol monomer molecules, 0.5 to 5 mol acid monomer molecules, and 0.1% to 100% of 0.5 to 5 mol emulsion and are crosslinked via condensation reactions to form a polymeric material.

20. A polymeric material according to claim 19, wherein the combination of alcohol monomer, acid monomer, and emulsion is heated.
